(19) United States
(12) Plant Patent Application Publication
(10) Pub. No.: US 2007/0226852 P1
(43) Pub. Date: Sep. 27, 2007

(54) ROSE PLANT NAMED 'POULPAH032'

(76) Inventor: Mogens N. Olesen, Fredensborg (DK)

Correspondence Address: POULSEN ROSER APS, 620 S. FRONT STREET, CENTRAL POINT, OR, 97502, US

(21) Appl. No.: 11/389,601

(22) Filed: Mar. 23, 2006

(51) Int. Cl. A01H 5/00

(52) U.S. Cl. PLT/140

(57) ABSTRACT

A new garden rose plant of the floribunda class which has abundant, dark red flowers and attractive foliage. This new and distinct variety has shown to be uniform and stable in the resulting generations from asexual propagation from vegetative cuttings and traditional budding.

### BOTANTICAL CLASSIFICATION

Rosa hybrid

### VARIETY DENOMINATION

‘Poulpah032’

### SUMMARY OF THE INVENTION

The present invention constitutes a new and distinct variety of garden rose plant that originated from a controlled crossing between the female seed parent, an unnamed seedling, and the male pollen parent, ‘Poulgrad’, described and illustrated in U.S. Plant Pat. No. 11,610. The two parents were crossed during the summer of 1997 and the resulting seeds were planted in a controlled environment in Fredensborg, Denmark. The new variety is named ‘Poulpah032’.

The new variety may be distinguished from the female parent by the following combination of characteristics:
1. Flowers of the seed parent have 50 petals on average. ‘Poulpah032’ has 75 to 80 petals.
2. The seed parent has dark pink flowers. ‘Poulpah032’ has dark red flowers.

The new variety may be distinguished from its male pollen parent, ‘Poulgrad’ by the following combination of characteristics:
1. ‘Poulgrad’ has 25 to 30 petals. ‘Poulpah032’ has 75 to 80 petals.
2. ‘Poulgad’ has a general tonality of flower color of Red (Group 53A to Red Group 46A. ‘Poulpah032’ is Red Group 53A.
3. Average flower diameter for ‘Poulgrad’ is 55 mm. Average flower diameter for ‘Poulpah032’ is 40 mm.

The objective of the hybridization of this rose variety was to create a new and distinct variety for garden use with unique qualities, such as:
1. Uniform and abundant dark red flowers;
2. Vigorous, but compact growth,
3. Superior disease resistance.

This combination of qualities is not present in previously available commercial cultivars of this type, known to the inventor, and distinguish ‘Poulpah032’ from all other varieties of which we are aware.

As part of the rose development program, Mogens N. Olesen germinated the seeds from the aforementioned hybridization during winter of 1997 and conducted evaluations on the resulting seedlings in a controlled environment in Fredensborg, Denmark.

‘Poulpah032’ was selected in the spring of 1998 by the inventor as a single plant from the progeny of the aforementioned hybridization.

Asexual reproduction of ‘Poulpah032’ by traditional budding and rooted cuttings was first done by Mogens N. Olesen in Fredensborg, Denmark in July 1998. This initial and other subsequent asexual propagations conducted in controlled environments have demonstrated that the characteristics of ‘Poulpah032’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, and stems, of ‘Poulpah032’. Specifically illustrated in the drawing:

FIG. 1A; Open flower;

FIG. 1B; Stem showing attachment of leaves and cluster of flowers;

FIG. 1C; Flower petals, detached;

FIG. 1D; Sepals, receptacle, and peduncle;

FIG. 1E; Bare stem exhibiting prickles;

FIG. 1F; Juvenile growth;

FIG. 1G; Upper surface of mature leaves.

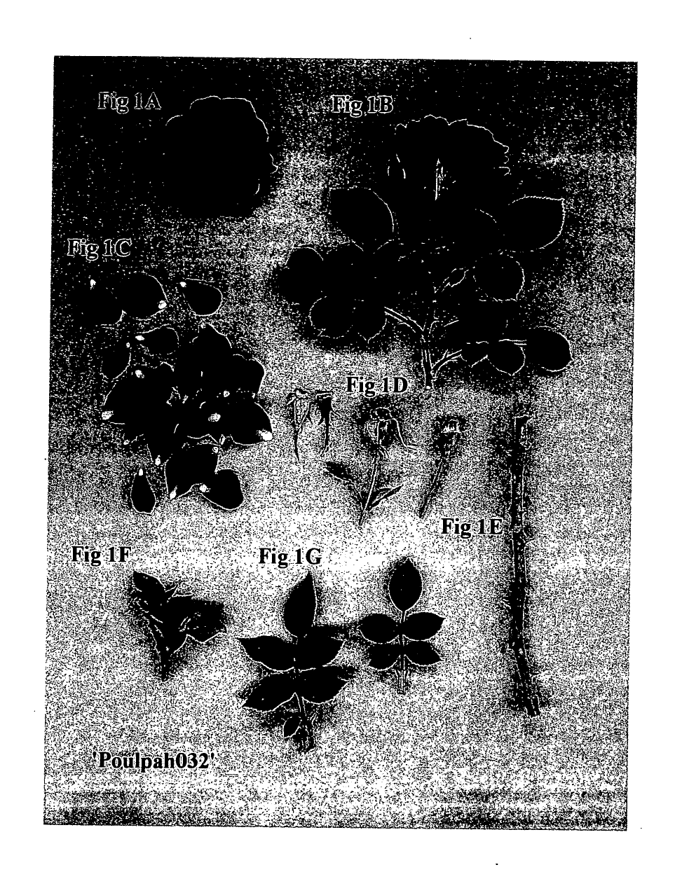

### DETAILED DESCRIPTION OF THE VARIETY

The following is a description of ‘Poulpah032’, observed in its growth in a field nursery in Jackson County, Oregon. Observed plants are 3 years of age, and were grown on Rosa multiflora understock. Color references are made using The Royal Horticultural Society (London, England) Colour Chart, 1995, except where common terms of color are used.

For a comparison, several physical characteristics of the rose variety ‘Poulhi016’, a rose variety from the same inventors described and illustrated in U.S. Plant Pat. No. 15,812, are compared to ‘Poulpah032’ in Chart 1.

CHART 1

| | ‘Poulpah032’ | ′Poulhi016′ |
|---|---|---|
| General tonality. | Red Group 53A | Red-Purple Group 60A |
| | | to Red Group 53A |
| Flower diameter. | 40 mm | 30 to 35 mm |
| Petalage. | 75 to 80 | 75 |

### FLOWER AND FLOWER BUD

Blooming habit: Continuous.
Flower bud:
   Size.—Upon opening, 20 mm in length from base of receptacle to end of bud. Bud diameter is 10 mm.
   Bud form.—Globular to ovoid.
   Bud color.—As sepals unfold, petals are Red-Purple Group 60A.
   Sepal inner surface.—Color: Yellow-Green Group 147C. Surface: Medium pubescence observed.
   Sepal outer surface.—Color: Yellow-Green Group 147C. Anthocyanic pigments the color of Greyed-Purple 183B observed. Texture: Smooth. Shape: Overall shape is subulate. Apex: Cirrhose. Base: Flat at union with receptacle. Margin: Margins have medium foliaceous appendages on three of the five sepals. Size: 20 mm (l)×6 mm (w).
   Receptacle.—Texture: Smooth, Shape- Urn-shaped. Size: 8 mm (h)×7 mm (w). Color: Yellow-Green Group 144A.
   Pedicel.—Surface: Rough with stipitate glands. Length: Normally 30 mm. Diameter: 2 mm. Color: Yellow-Green Group 144A. Anthocyanic pigments are very weak Greyed-Purple 183C. Strength: Somewhat strong.
Flower bud development: Flower buds are borne in clusters of 3 to 5 flower buds per stem.
Flower bloom:
   Duration.—The blooms have a duration on the plant of approximately 15 days. Petals fall cleanly away from plant after flowers have fully matured. Size: Flower diameter is 40 mm when fully open. Flower depth is 15 mm.
   Flower shape.—General shape is a rosette with many slightly overlapping petals of different sizes.
   Shape of flower, side view.—Upon opening: Upper portion: Flattened convex. Lower portion: Flat. After opening: Upper portion: Flattened convex. Lower portion: Concave.
   Petalage.—Under normal conditions, flowers have 75 to 80 petals total, 10 of which are petaloids.
Flower petal coloration:
   Upon opening, outer petals.—
   Upper surface.—Red Group 53A and Red-Purple Group 59A blended. Occasionally petals exhibit streaks the color of white group 155A.
   Lower surface.—Red-Purple Group 59B.
   Upon opening, inner petals.—
   Upper surface.—Red Group 53A and Red-Purple Group 59A blended.
   Lower surface.—Red-Purple Group 59B.
   Basal petal spots, upon opening.—
   Upper surface.—Green-White Group 157D.
   Lower surface.—Green-White Group 157D.
   After opening, outer petals.—
   Upper surface.—Red Group 53A and Red-Purple Group 60A, blended. Occasionally petals exhibit streaks the color of white group 155A.
   Lower surface.—Red-Purple Group 60A.
   After opening, inner petals.—
   Upper surface.—Red Group 53A and Red-Purple Group 60A blended.
   Lower surface.—Red-Purple Group 60A.
   Basal petal spots, after opening.—
   Upper surface.—Yellow-Green Group 149D to Yellow Group 2D.
   Lower surface.—Yellow-Green Group 149D to Yellow Group 2D.
   General tonality of flower.—On open flowers, Red Group 53A. No change in the general tonality at the end of the 10^{th} day.
Petals:
   Petal reflex.—Somewhat reflexed.
   Margin.—Entire and uniform.
   Shape.—Narrow elliptic. Apex: Cuspidate. Base: Acute.
   Size.—23 mm (l)×18 mm (w).
   Texture.—Smooth.
   Thickness.—Thin.
   Arrangement.—Not Formal.
Petaloids:
   Shape.—Elliptic.
   Color.—Upper surface is a blend of Red Group 53A and Red-Purple Group 60A. Lower surface is Red-Purple Group 60A. Base of petaloids are Yellow-Green Group 149D to Yellow Group 2D.
   Size.—Normally 5 mm (l)×4 mm (w).
Reproductive organs:
   Pollen.—None observed.
   Anthers.—Size: 2 to 3 mm in length. Color: Yellow-Orange Group 17A. Quantity: 34 on average.
   Filaments.—Color: Yellow Group 8B. Length: 3 mm.
   Pistils.—Length: 3 nm. Quantity: 57 normally.
   Stigmas.—Inferior in location relative to the length of the filaments and the height of the anthers. Color: Greyed-Yellow 160D.
   Styles.—Color: Red-Purple Group 59A. Hips: None observed.

### PLANT

Plant growth: Upright and very bushy. When grown as a budded field grown plant on Rosa multiflora understock, plants achieve 70 to 100 cm in height. Average spread is 80 cm.
Stems:
   Color.—Juvenile: Yellow-Green Group 144B. Mature: Yellow-Green Group 146A.
   Length.—Canes are normally 40 to 55 cm from the base of the plant to the flowering portion.
   Stem diameter.—Normally 4 mm.
   Internodes.—On mature canes, there is an average distance of 27 mm between nodes.
   Surface texture.—Young wood: Smooth. Older wood: Smooth.
Prickles:
   Incidence.—Normally 10 prickles per 10 cm of stem.
   Size.—Average length of prickles on mature stems is 6 mm.
   Shape.—Upper side: Convex. Lower side: Concave.
   Color.—Juvenile prickles are Yellow-Green Group 146D with intonations of Greyed-Purple Group 184A.
Plant foliage: Normal number of leaflets leaves in middle of the stem: 7 leaflets.
   Compound leaf.—On average, 95 mm (l)×52 mm (w).
   Quantity.—3 leaves per 10 cm of stem on average.
   Color of mature foliage.—Upper side: Yellow-Green Group 147A. Lower side: Yellow-Green Group 147B.
   Color of juvenile foliage.—Upper side: Yellow-Green Group 144A with Greyed-Red Group 178A on leaf margins. Lower side: Yellow-Green Group 144B.
Plant leaves and leaflets:
   Stipules.—Size: 20 mm in length. Shape: Linear, slightly broad based with outward extending apecies. Margins: Finely serrated with stipitate glands at margins. Color: Yellow-Green Group 146B.
   Petiole.—Length: 20 mm on average. Diameter: Normally 1.5 mm.
   Upper surface.—Color: Yellow-Green Group 146A.
   Lower surface.—Color: Yellow-Green Group 146B. Observations: Smooth.
   Rachis.—Length: Normally 45 mm. Upper surface: Color: Yellow-Green Group 146A.
   Lower surface.—Color: Yellow-Green Group 146A. Observations: Smooth.
   Leaflet.—Edge: Serrated. Size: Average size of the terminal leaflet is 31 mm(l)×20mm(w). Shape: Ovate. Base: Obtuse. Apex: Acuminate. Texture: Smooth. Thickness: Average. Arrangement: Odd pinnate. Venation: Reticulate. Glossiness: Somewhat glossy.
Disease resistance: Above average resistance to powdery and downy mildew, rust, black spot, and Botrytis under normal growing conditions in Jackson County, Oregon.
Cold Hardiness: The variety ‘Poulpah032’ has been found to be cold tolerant to USDA Cold Hardiness Zone 6.

1. A new and distinct variety of rose plant of the floribunda rose class named ‘Poulpah032’, substantially as illustrated and described herein due to its abundant dark red flowers, disease resistance, and extended period of bloom.
